(19) United States
(12) Patent Application Publication
(10) Pub. No.: US 2006/0293378 A1
(43) Pub. Date: Dec. 28, 2006

(54) Method of lowering intraocular pressure

(76) Inventors: Gregory McIntire, Rochester, NY (US); Bruce Pfister, Rochester, NY (US); Ruiwen Shi, Cupertino, CA (US); Stephen Bartels, Pittsford, NY (US)

Correspondence Address: Bausch & Lomb Incorporated, One Bausch & Lomb Place, Rochester, NY, 14604-2701, US

(21) Appl. No.: 11/475,587

(22) Filed: Jun. 27, 2006

Related U.S. Application Data
(60) Provisional application No. 60/694,591, filed on Jun. 28, 2005.

(51) Int. Cl. A61K 31/407

(52) U.S. Cl. 514/410

(57) ABSTRACT

A method of lowering intraocular pressure and/or treating glaucoma comprises administering to a patient a therapeutically effective amount of a fused pyrrolocarbazole derivative, or a pharmaceutically acceptable salt or prodrug thereof.

### CROSS REFERENCE

This application claims the benefit of Provisional Patent Application No. 60/694,591 filed Jun. 28, 2005 and is incorporated herein by reference.

### FIELD

This invention relates to a method of lowering intraocular pressure.

### BACKGROUND

The term ocular hypertension usually refers to any situation in which the pressure inside the eye, called intraocular pressure, is higher than normal. Eye pressure is measured in millimeters of mercury (mm Hg). Normal eye pressure ranges from 10-21 mm Hg. Ocular hypertension is an eye pressure of greater than 21 mm Hg. Studies estimate that 3-6 million people in the United States alone, including 4-10% of the population older than 40 years, have intraocular pressures of 21 mm Hg or higher.

Elevation of intraocular pressure has been considered one of the major causes for glaucoma, a progressive blinding disease affecting about 3 million Americans. Higher intraocular pressure is believed to cause damage to the optic nerves and retina and result in degeneration of retinal ganglion cells and blindness. Over a 5-year period, several studies have shown the incidence of glaucomatous damage in people with ocular hypertension to be about 2.6-3% for intraocular pressures of 21-25 mm Hg, 12-26% for intraocular pressures of 26-30 mm Hg, and approximately 42% for those higher than 30 mm Hg.

Treatment of glaucoma includes pharmaceutical agents, laser surgery and incisional surgery. Pharmaceutical agents currently used for treating glaucoma associated with elevated intraocular pressure include adrenergic agonists (such as epinephrine, dipervefrin and apraclonidine), cholinergic agonists (such as pilocarpine), beta-blockers (such as betaxolol, levobunolol, and timolol), carbonic anhydrase inhibitors (such as acetazolamide) or prostaglandin analogues (such as latanoprost and bimatoprost) and alpha-adrenergic agonists (such as brimonidine). These agents help restore the intraocular pressure to normal levels either by inhibiting the production of aqueous humor by the ciliary body or facilitating trabecular or uveoscleral aqueous humor outflow.

However, in view of the prevalence of ocular hypertension, there is a continuing need for new pharmaceutical agents capable of lowering intraocular pressure.

It has been know for some time that tyrosine kinase inhibitors can be used potentially to treat eye disease. U.S. Pat. No. 5,980,929, U.S. Pat. No. 5,919,813 and International Publication No. WO 00/67,738 disclose the use of genestien as a protein tyrosine kinase pathway inhibitor in the treatment of retinal ischemia, diabetic retinopathy, ocular inflammation, age-related macular degeneration and other ocular disorders. Each of these patents discuss administration by injection in addition to other systemic forms of administration.

Various synthetic small organic molecules that are biologically active and generally known in the art as “fused pyrrolocarbazoles” have been prepared. Examples of such patents include U.S. Pat. Nos. 5,475,110, 5,591,855, 5,594,009, 5,616,724, 5,705,511 and 6,200,968. The fused pyrrolocarbazoles have been disclosed for use in a variety of treatments, including inhibition of protein kinase C (“PKC”), inhibition of trk tyrosine kinase activity and inhibition of the cellular pathways involved in the inflammation process.

Certain selected fused pyrrolocarbazoles are taught in U.S. Pat. No. 6,630,500 to have activity for inhibition of VEGFR2 as a potential therapeutic for treatment of ocular disease such as retinopathy (including diabetic retinopathy), edema (including macular edema) and ocular inflammation.

U.S. Patent Application Publication No. 2003/0181504 discloses that certain fused naphthylazoles and indenoazoles, such as substituted 2-(benzo[g]indazol-1-yl)-ethylamines and 1-(4H-indeno[1,2-c]pyrazol-1-yl)-1-methylethylamines, exhibit activity for lowering and controlling normal or elevated intraocular pressure and for treating glaucoma.

### SUMMARY

In one aspect, the invention resides in a method of controlling normal or elevated intraocular pressure, comprising administering to a patient a therapeutically effective amount of a fused pyrrolocarbazole having the formula I, or a pharmaceutically acceptable salt or prodrug thereof: 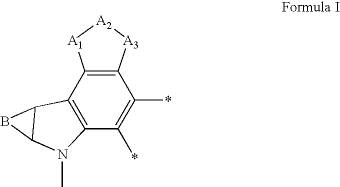
wherein at least one of A1, A2 or A3 is a nitrogen atom; B is an aryl or heteroaryl ring system and the designation * indicates the attachment point of an additional fused ring system. N— is a substituted nitrogen atom. By way of example, the nitrogen is substituted with —H, —H₂⁺ or any other substituent group.

Typically, the fused pyrrolocarbazole has the formula II. 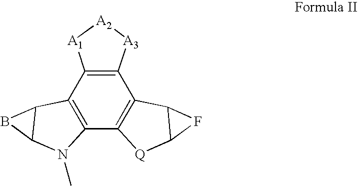
wherein at least one of A1, A2 and A3 is a nitrogen atom; each of B1 and F1 independently forms an aryl or heteroaryl ring; and Q is a moiety containing one or more nitrogen atoms or carbon atoms. In one embodiment, Q is a substituted or unsubstituted nitrogen atom and the structure of formula II is an indolocarbazole. In another embodiment, Q is a substituted or unsubstituted carbon atom and the structure of formula II is an indenocarbazole. N— is a substituted nitrogen atom. By way of example, the nitrogen is substituted with —H, —H₂⁺ or any other substituent group.

In another aspect, the invention resides in a method of controlling normal or elevated intraocular pressure, comprising administering to a patient a therapeutically effective amount of an indenocarbazole derivative having the formula III, or a pharmaceutically acceptable salt or prodrug thereof: 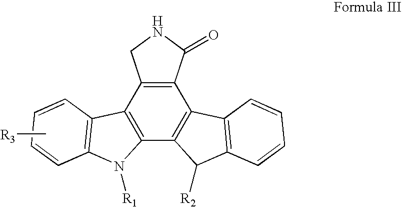
wherein:

R1 and R2 are the same or different and are independently selected from H, or alkyl of 1-8 carbons, preferably an alkyl of 1-4 carbons, substituted with OH, or —OR4 where R4 is an alkyl of 1-4 carbons, aryl of 6-12 carbons, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein

R5 is alkyl of 1-4 carbons or aryl of 6-12 carbons, preferably phenyl or naphthyl;

R6 is H, alkyl of 1-4 carbons, aryl of 6-10 carbons, preferably phenyl or naphthyl, or heteroaryl of 5-12 ring members;

R7 is H or alkyl of 1-4 carbons;

R8 is the residue of an amino acid after the hydroxyl group of the carboxyl group is removed;

n is an integer of 1-4; and

y is 1 or 2.

In one embodiment, said indenocarbazole derivative has the formula IV 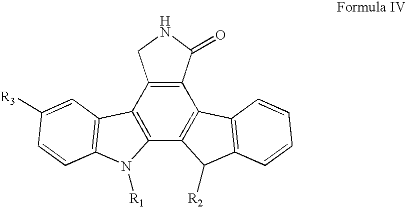
wherein R1, R2 and R3 have the meanings given in the preceding paragraph.

Conveniently, R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)yR6 or —SR8; wherein R5 is alkyl of 1-4 carbons or aryl of 6-12 carbons; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

Preferably, R1 is —CH₂CH₂CH₂OH or —CH₂CH₂CH₂OCOCH₂N(CH₃)₂; R2 is H; and R3 is —CH₂OR7; wherein R7 is alkyl of 1-4 carbons, and especially is iso-propyl.

In a further aspect, the invention resides in a method of treating glaucoma, comprising administering to a patient a therapeutically effective amount of an indenocarbazole derivative having the formula III, or a pharmaceutically acceptable salt or prodrug thereof: 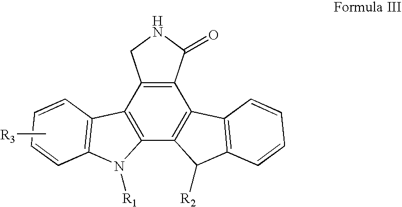
wherein:

R1 and R2 are the same or different and are independently selected from H, or alkyl of 1-8 carbons, preferably an alkyl of 1-4 carbons, substituted with OH, or —OR4 where R4 is an alkyl of 1-4 carbons, aryl of 6-12 carbons, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SOYR6 or —SR6; and wherein

R5 is alkyl of 1-4 carbons or aryl of 6-12 carbons, preferably phenyl or naphthyl;

R6 is H, alkyl of 1-4 carbons, aryl of 6-10 carbons, preferably phenyl or naphthyl, or heteroaryl of 5-12 ring members;

R7 is H or alkyl of 1-4 carbons;

R8 is the residue of an amino acid after the hydroxyl group of the carboxyl group is removed;

n is an integer of 1-4; and

y is 1 or 2.

### BRIEF DESCRIPTION OF THE DRAWINGS

FIGS. 1 to 3 are graphs showing the effect on intraocular pressure over time of injecting various levels of a fused pyrrolocarbazole according to one example of the invention into the right eyes of rabbits versus untreated left eyes.

### DETAILED DESCRIPTION OF THE EMBODIMENTS

As used herein the term “fused pyrrolocarbazole” is intended to refer to any of the general family of compounds having the core structure of formula I or formula II as well as any of the more specific series of compounds having the structure of either formula III or formula IV.

As used herein the term “prodrug” is intended to include any covalently bonded carrier, which releases the active parent pharmaceutical agent as a compound of the present invention in vivo when such prodrug is administered to a mammalian subject. Since prodrugs are known to enhance numerous desirable qualities of pharmaceuticals (e.g., solubility, bioavailability, manufacturing, etc.) the compounds of the present invention may be delivered in prodrug form. Thus, the present invention contemplates prodrugs of the compounds of the present invention, compositions containing the same and methods of treating diseases and disorders with such prodrugs. Prodrugs of a compound of the present invention, for example Formula III, may be prepared by modifying functional groups present in the compound in such a way that the modifications are cleaved, either in routine manipulation or in vivo, to the parent compound. Accordingly, prodrugs include, for example, compounds of the present invention wherein a hydroxy, amino, or carboxy group is bonded to any group that, when the prodrug is administered to a mammalian subject, cleaves to form a free hydroxyl, free amino, or carboxylic acid, respectively. Examples include, but are not limited to, the residue of an amino acid after the hydroxyl group of the carboxyl group is removed acetate, formate and benzoate derivatives of alcohol and amine functional groups; and alkyl, carbocyclic, aryl and alkylaryl esters such as methyl, ethyl, propyl, iso-propyl, butyl, isobutyl, sec-butyl, tert-butyl, cyclopropyl, phenyl, benzyl and phenethyl esters and the like.

The present invention provides a method of controlling normal or elevated intraocular pressure and/or of treating glaucoma in a patient by administering to the patient a therapeutically effective amount of certain fused pyrrolocarbazoles or pharmaceutically acceptable salts or prodrugs thereof. In general effective fused pyrrolocarbazoles have a core structure with the formula I: 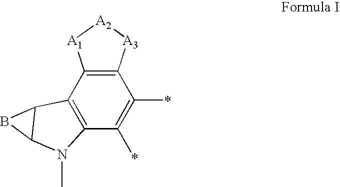
wherein at least one of A1, A2 or A3 is a nitrogen atom; B is an aryl or heteroaryl ring system, such as a benzene ring, and the designation * indicates the attachment point of an additional fused ring system. N— is a substituted nitrogen atom. By way of example, the nitrogen is substituted with —H, —H₂⁺ or any other substituent group.

It is to be appreciated that the core structures provided herein are presented by way of the general guidance and are not to be taken as limiting the scope of the invention. For example, certain cores indicate the presence of certain atoms for illustrative purposes. It will be appreciated that such atoms may be bonded to additional groups, or may be further substituted without deviating from the spirit of the invention.

More particularly, the fused pyrrolocarbazoles useful in the method of the invention have a core structure with the formula II: 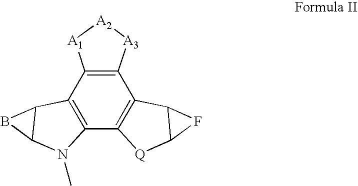
wherein at least one of A1, A2 and A3 is a nitrogen atom; each of B1 and F1 independently forms an aryl or heteroaryl ring, such as a benzene ring; and Q is a moiety containing one or more nitrogen atoms or carbon atoms. In one embodiment, Q is a substituted or unsubstituted nitrogen atom and the structure of formula II is an indolocarbazole. In another embodiment, Q is a substituted or unsubstituted carbon atom and the structure of formula II is an indenocarbazole. N— is a substituted nitrogen atom. By way of example, the nitrogen is substituted with —H, —H₂⁺ or any other substituent group.

In one embodiment, the method of the invention comprises administering to a patient suffering from elevated optical pressure and/or glaucoma a therapeutically effective amount of an indenocarbazole derivative having the formula III, or a pharmaceutically acceptable salt or prodrug thereof: 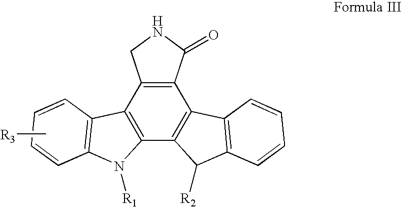
wherein:

R1 and R2 are the same or different and are independently selected from H, or alkyl of 1-8 carbons, preferably an alkyl of 1-4 carbons, substituted with OH, or —OR4 where R4 is an alkyl of 1-4 carbons, aryl of 6-12 carbons, preferably phenyl or naphthyl, or the residue of an amino acid after the hydroxyl group of the carboxyl group is removed; and

R3 is —CH₂OH; —CH₂OR7; —(CH₂)ₙSR5; —(CH₂)ₙSO_{y}R5; —CH₂SR₅; or alkyl of 1-8 carbons, preferably an alkyl of 1-4 carbons, substituted with —OH, —OR5, —OR8, —CH₂OR7, —SO_{y}R6 or —SR6; and wherein

R5 is alkyl of 1-4 carbons or aryl of 6-12 carbons, preferably phenyl or naphthyl;

R6 is H, alkyl of 1-4 carbons, aryl of 6-10 carbons, preferably phenyl or naphthyl, or heteroaryl of 5-12 ring members;

R7 is H or alkyl of 1-4 carbons;

R8 is the residue of an amino acid after the hydroxyl group of the carboxyl group is removed;

n is an integer of 1-4; and

y is 1 or 2.

Preferably, said indenocarbazole derivative has the formula IV 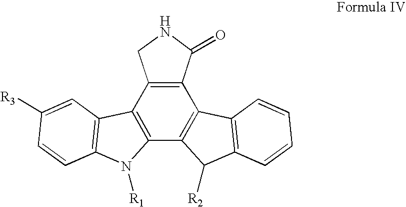
wherein R1, R2 and R3 have the meanings given in the preceding paragraph.

Conveniently, in formula III and IV, R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)_{y}R6 or —SR8; wherein R5 is alkyl of 1-4 carbons or aryl of 6-12 carbons; R6 is H, alkyl of 1-4 carbons or aryl of 6-10 carbons; R7 is H or alkyl of 1-4 carbons; and R8 is the residue of an amino acid after the hydroxyl group of the carboxyl group is removed.

Preferably, R1 is —CH₂CH₂CH₂OH or —CH₂CH₂CH₂OCOCH₂N(CH₃)₂; R2 is H; and R3 is —CH₂OR7; wherein R7 is alkyl of 1-4 carbons, and especially is iso-propyl.

Examples of suitable indenocarbazole derivatives of the formula III are shown in Table 1 below:

TABLE 1

| Compound | R1 | R2 | R3 |
|---|---|---|---|
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O—CH(CH₃)CH₂CH₃ |
| 4 | —CH₂CH₂CH₂OH | —H | (S)—CH₂O—CH(CH₃)CH₂CH₃ |
| 5 | —CH₂CH₂CH₂OH | —H | (R)—CH₂O—CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —CH₂CH₂CH₂OH | —H | —CH₂OCH₂CH₃ |
| 14 | —CH₂CH₂CH₂OH | —H | —CH(CH₃)O—CH₂CH₂CH₂CH₃ |
| 15 | —CH₂CH₂CH₂OH | —H | —CH(CH₃)O—CH(CH₃)₂ |
| 16 | —CH₂CH₂CH₂OH | —H | —CH₂OC(CH₃)₃ |
| 17 | —CH₂CH₂CH₂OCO—CH₂NH₂ | —H | —CH₂OCH(CH₃)₂ |
| 18 | —CH₂CH₂CH₂OCOCH₂—NH₂CH₂CH₂CH₂CH₂NH₂ | —H | —CH₂OCH(CH₃)₂ |
| 19 | CH₂CH₂CH₂OCOCH₂—CH₂NH₂ | —H | —CH₂OCH(CH₃)₂ |
| 20 | CH₂CH₂CH₂OCOCH₂—CH₂CH₂N(CH₃)₂ | —H | —CH₂OCH(CH₃)₂ |
| 21 | CH₂CH₃CH₃OCO—CH₂N(CH₂)₂ | —H | —CH₂OCH(CH₃)₂ |
| 22 | —CH₂CH₂CH₂OCO—CH₂CH₂CH₂ | —H | —CH₂OCH(CH₃)₂ |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−)—CH(OCH₃)CH₃ |
| 34 | —CH₂CH₂CH₂OH | —CH₂OH | CH₂OCH(CH₃)₂ |

Particularly preferred indenocarbazole derivatives are the compounds 2 and 21 in Table 1, in which R3 group is located at the 9-position on the carbazole ring as shown in Formula IV, namely 3-[5,6,7,13-tetrahydro-9-[(1-methylethoxy)methyl]-5-oxo-12H-indeno(2,1-a)pyrrolo(3,4-c)carbazol-12-yl)propyl ester (compound 2) and its N,N-dimethylglycine ester (compound 21):

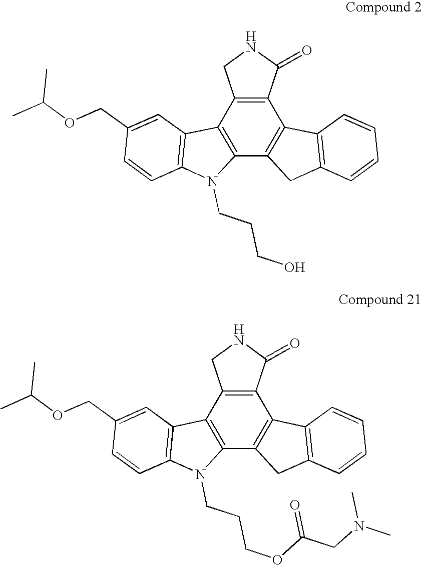

Pharmaceutically acceptable salts of the fused pyrrolocarbazoles described herein are also useful in the treatment methods of the present invention. The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine.

The fused pyrrolocarbazoles employed herein may be synthesized in a number of ways well known to those skilled in the art. By way of example, suitable synthesis techniques are disclosed in U.S. Pat. Nos. 5,475,110, 5,591,855, 5,594,009, 5,616,724, 5,705,511 and 6,630,500, each of which is incorporated herein by reference in its entirety.

It will be appreciated that the compounds of the present invention may contain one or more asymmetrically substituted carbon atoms and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare such optically active forms. For example, mixtures of stereoisomers may be separated by standard techniques including, but not limited to, resolution of racemic forms, normal, reverse-phase and chiral chromatography, preferential salt formation, recrystallization and the like, or by chiral synthesis either from active starting materials or by deliberate chiral synthesis of target centers.

As will be readily understood, functional groups present on the compounds of the present invention may contain protecting groups. For example, the amino acid side chain substituents of the compounds can be substituted with protecting groups such as benzyloxycarbonyl or t-butoxycarbonyl groups. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl (Cbz; Z) group and the tert-butyloxycarbonyl (Boc) group. Other preferred protecting groups according to the invention may be found in Greene, T. W. and Wuts, P. G. M., “Protective Groups in Organic Synthesis” 2d. Ed., Wiley & Sons, 1991.

The fused pyrrolocarbazoles disclosed herein have been found to be effective in the treatment of elevated intraocular pressure and/or glaucoma in warm-blooded animals and can be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), alpha1 antagonists (e.g., nipradolol), alpha2 agonists (e.g. iopidine and brimonidine), miotics (e.g., pilocarpine and epinephrine), prostaglandin analogs (e.g., latanoprost, travaprost, unoprostone, and compounds set forth in U.S. Pat. Nos. 5,889,052; 5,296,504; 5,422,368; and 5,151,444), “hypotensive lipids” (e.g., lumigan and compounds set forth in U.S. Pat. No. 5,352,708), and neuroprotectants (e.g., compounds from U.S. Pat. No. 4,690,931).

The fused pyrrolocarbazoles disclosed herein can be incorporated into various types of ophthalmic formulations including, but not limited to, solutions, emulsions, suspensions, ointments, tablets, capsules and implants. The formulations can be administered to a subject by any method known in the art including, but not limited to, by injection intravenously, intramuscularly or subcutaneously, by intravitreous injection/implantation, topical application or intraocular implantation.

In producing ophthalmic formulations, the fused pyrrolocarbazoles may be combined with ophthalmologically acceptable preservatives, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity, such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum.

For example, where the fused pyrrolocarbazoles are formulated as topical ophthalmic suspensions or solutions, the compounds will normally be contained in these formulations in an amount up to about 20% by weight, such as about 0.01% to about 5% by weight, with the formulation having a pH of about 5 to about 8. For topical presentation to a patient, different amounts of these formulations would then be delivered to the surface of the eye multiple times per day according to the discretion of a skilled clinician.

In one embodiment, the fused pyrrolocarbazoles disclosed herein form part of a pharmaceutical delivery system also comprising a delivery device or a biodegradable or drug-eluting polymer matrix that is sized and configured to be inserted into the eye of a patient. Such a delivery system provides for controlled and prolonged exposure of the patient to the ophthalmologically effective component without the concentration gradients and other side effects frequently encountered with bolus injections.

Where the pharmaceutical delivery system comprises a delivery device sized and configured to be inserted into the eye of a patient, the delivery device conveniently comprises a reservoir having a drug impermeable portion that surrounds at least a portion of a drug core comprising the fused pyrrolocarbazole and that restricts flow of fused pyrrolocarbazole from the reservoir. The drug impermeable portion would typically be made of silicone. The reservoir could also include a drug permeable portion in the form of a wall, layer or coating formed of a drug permeable portion, such as a polyinyl alcohol, or a permeable plug covering in an opening in the reservoir. Alternatively, the size and shape of the opening in the reservoir could be arranged to provide the desired controlled release of the fused pyrrolocarbazole.

Where the pharmaceutical delivery system comprises a biodegradable polymer matrix, suitable biodegradable polymers include polymers of hydroxyaliphatic carboxylic acids, such as polycaprolactone, polylactic acid or polylactide, polyglycolic acid or polyglycolide, and copolymers thereof, polyanhydrides, poly(ortho esters), and polysaccharides. Typically, the amount of fused pyrrolocarbazole present in the biodegradable polymer matrix is between about 0.1 wt. % and about 60 wt. %, such as about 1 wt % to about 50 wt %, based upon the total weight of the biodegradable polymer matrix. The biodegradable polymer matrices of the invention are generally formulated with particles of the fused pyrrolocarbazole mixed within the bioerodible polymer matrix. Release of the agent is achieved by erosion of the polymer followed by exposure of previously entrapped agent particles to the vitreous and subsequent dissolution and release of agent.

Where the pharmaceutical delivery system comprises a drug eluting polymer matrix, the drug eluting polymer is conveniently made from siloxane copolymer, such as a fluorinated side-chain polysiloxane optionally polymerized with a comonomer such as methyl methacrylate, N,N-dimethylacrylamide, acrylamide, N-methylacrylamide, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, N-vinylpyrrolidione and hydroxybutyl methacrylate. By varying the concentration of the hydrophobic siloxane backbone, the polar —CF₂—H tail and any comonomer(s), if used, the hydrophobic/hydrophilic balance and hence the pharmaceutical agent release characteristics of the polymer can controlled.

The invention will now be more particularly described with reference to the following Examples.

### EXAMPLE 1

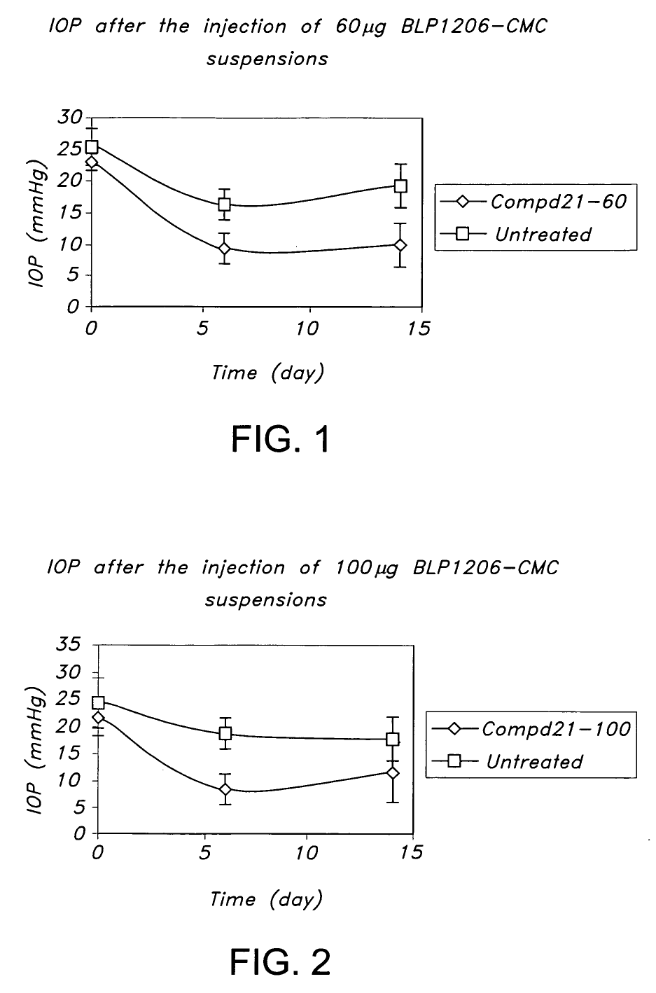

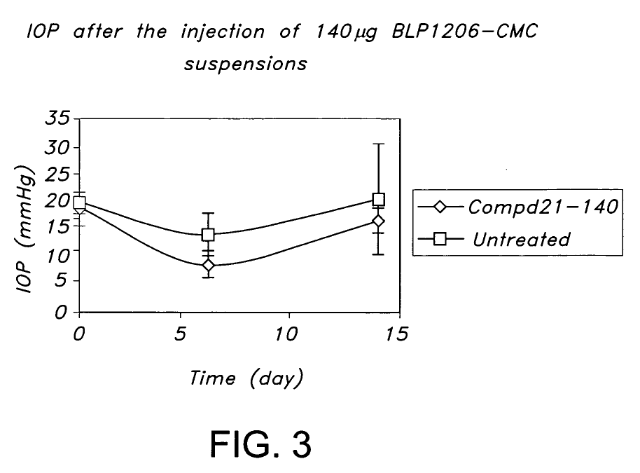

An aqueous suspension was produced containing 2 mg/mL of compound 21 illustrated above together with 7.5 mg/mL of sodium carboxymethylcellulose. The suspension was injected into the right eyes of rabbits in amounts of 30 μL, 50 μL and 70 μL to give dosages of 60 μg, 100 μg and 140 μg respectively of compound 21. The intraocular pressure of the rabbits after being injected with the suspensions as compared to the intraocular pressure of untreated left eyes is shown in FIGS. 1 to 3. From these graphs it will be seen that the treatment method of the invention resulted in a significantly increased lowering of intraocular pressure, which was maintained over the 14 days of the test.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What we claim is:

1. A method of controlling normal or elevated intraocular pressure, comprising administering to a patient a therapeutically effective amount of a fused pyrrolocarbazole having the formula I, or a pharmaceutically acceptable salt or prodrug thereof: 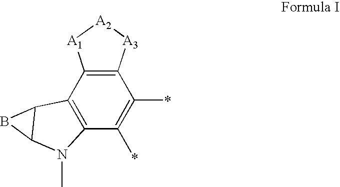
wherein at least one of A1, A2 or A3 is a nitrogen atom; B is an aryl or heteroaryl ring system and the designation * indicates the attachment point of an additional fused ring system; and N— is a substituted nitrogen.

2. The method of claim 1 wherein the fused pyrrolocarbazole has the formula II 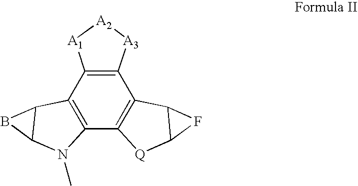
wherein at least one of A1, A2 and A3 is a nitrogen atom; each of B1 and F1 independently forms an aryl or heteroaryl ring; Q is a moiety containing one or more nitrogen atoms or carbon atoms; and N— is a substituted nitrogen.

3. The method of claim 2 wherein Q is a substituted or unsubstituted nitrogen atom.

4. The method of claim 2 wherein Q is a substituted or unsubstituted carbon atom.

5. The method of claim 2 wherein each of B1 and F1 independently forms a benzene ring.

6. The method of claim 1 wherein the fused pyrrolocarbazole forms part of a pharmaceutical composition comprising a pharmaceutical solution or suspension, a delivery device, or a drug-eluting polymer matrix that is sized and configured to be injected or inserted into the eye of a patient.

7. A method of controlling normal or elevated intraocular pressure, comprising administering to a patient a therapeutically effective amount of an indenocarbazole derivative having the formula III, or a pharmaceutically acceptable salt or prodrug thereof: 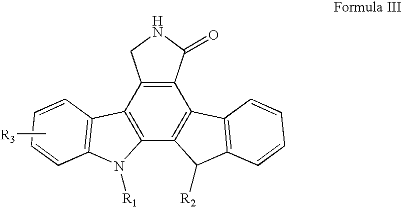
wherein:
R1 and R2 are the same or different and are independently selected from H, or alkyl of 1-8 carbons, substituted with OH, or —OR4 where R4 is an alkyl of 1-4 carbons, aryl of 6-12 carbons; and
R3 is —CH₂OH; —CH₂OR7; —(CH₂)ₙSR5; —(CH₂)ₙSO_{y}R5; —CH₂SR₅; or alkyl of 1-8 carbons, substituted with —OH, —OR5, —OR8, —CH₂OR7, —SO_{y}R6 or —SR6; and wherein
R5 is alkyl of 1-4 carbons or aryl of 6-12 carbons;
R6 is H, alkyl of 1-4 carbons, aryl of 6-10 carbons or heteroaryl of 5-12 ring members;
R7 is H or alkyl of 1-4 carbons;
R8 is the residue of an amino acid after the hydroxyl group of the carboxyl group is removed;
n is an integer of 1-4; and
y is 1 or 2.

8. The method of claim 7 wherein said indenocarbazole derivative has the formula IV: 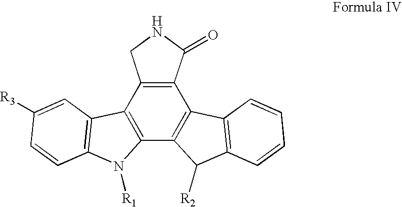
wherein R1, R2 and R3 have the meanings given in claim 1.

9. The method of claim 8, wherein R1 is an alkyl of 1-4 carbons, substituted with —OH or —OR4 wherein R4 is the residue of an amino acid after the hydroxyl group of the carboxyl group is removed; R2 is H; and R3 is alkyl of 1-4 carbons, substituted with —OR5, —OR8, —CH₂OR7, —S(O)_{y}R6 or —SR8; and wherein R5 is alkyl of 1-4 carbons or aryl; R6 is H, alkyl of 1-4 carbons or aryl of 6-10 carbons or heteroaryl of 5-12 ring members; R7 is H or alkyl of 1-4 carbons; and R8 is the residue of an amino acid after the hydroxyl group of the carboxyl group is removed.

10. The method of claim 9, wherein R1 is —CH₂CH₂CH₂OH or —CH₂CH₂CH₂OCOCH₂N(CH₃)₂; R2 is H; and R3 is —CH₂OR7 wherein R7 is alkyl of 1-4 carbons.

11. The method of claim 10, wherein R7 is iospropyl.

12. The method of claim 7, wherein the fused indenocarbazole is selected from the compounds represented in the following table, pharmaceutically acceptable salts thereof and/or prodrugs thereof:

| Compound | R1 | R2 | R3 |
|---|---|---|---|
| 1 | —CH₂CH₂CH₂OH | —H | —CH₂OCH₃ |
| 2 | —CH₂CH₂CH₂OH | —H | —CH₂OCH(CH₃)₂ |
| 3 | —CH₂CH₂CH₂OH | —H | —CH₂O—CH(CH₃)CH₂CH₃ |
| 4 | —CH₂CH₂CH₂OH | —H | (S)—CH₂O—CH(CH₃)CH₂CH₃ |
| 5 | —CH₂CH₂CH₂OH | —H | (R)—CH₂O—CH(CH₃)CH₂CH₃ |
| 6 | —CH₂CHOHCH₃ | —H | —CH₂OCH₂CH₃ |
| 7 | —CH₂CH₂CH₂OH | —H | —CH₂OCH₂CH₂CH₃ |
| 8 | —CH₂CH₂CH₂OH | —H | —CH₂OCH₂CH₂CH₂CH₃ |
| 9 | —CH₂CH₂CH₂OH | —H | —CH(CH₃)OCH₂CH₃ |
| 10 | —CH₂CH₂CH₂OH | —H | (chiral) |
| | | | —CH(CH₃)OCH₂CH₃ |
| 11 | —CH₂CH₂CH₂OH | —H | (chiral) |
| | | | —CH(CH₃)OCH₂CH₃ |
| 12 | —CH₂CH₂CH₂OH | —H | —CH(CH₃)OCH₃ |
| 13 | —CH₂CH₂CH₂OH | —H | —CH₂OCH₂CH₃ |
| 14 | —CH₂CH₂CH₂OH | —H | —CH(CH₃)O—CH₂CH₂CH₂CH₃ |
| 15 | —CH₂CH₂CH₂OH | —H | —CH(CH₃)O—CH(CH₃)₂ |
| 16 | —CH₂CH₂CH₂OH | —H | —CH₂OC(CH₃)₃ |
| 17 | —CH₂CH₂CH₂OCO—CH₂NH₂ | —H | —CH₂OCH(CH₃)₂ |
| 18 | —CH₂CH₂CH₂OCOCH₂—NH₂CH₂CH₂CH₂CH₂NH₂ | —H | —CH₂OCH(CH₃)₂ |
| 19 | CH₂CH₂CH₂OCOCH₂—CH₂NH₂ | —H | —CH₂OCH(CH₃)₂ |
| 20 | CH₂CH₂CH₂OCOCH₂—CH₂CH₂N(CH₃)₂ | —H | —CH₂OCH(CH₃)₂ |
| 21 | CH₂CH₃CH₃OCO—CH₂N(CH₂)₂ | —H | —CH₂OCH(CH₃)₂ |
| 22 | —CH₂CH₂CH₂OCO—CH₂CH₂CH₂ | —H | —CH₂OCH(CH₃)₂ |
| 23 | —CH₂CH₂OH | —H | —CH₂SCH₂CH₃ |
| 24 | —CH₂CH₂CH₂OH | —H | —CH₂SCH₂CH₃ |
| 25 | —CH₂CH₂CH₂OH | —H | —CH₂S(O)CH(CH₃)₂ |
| 26 | —CH₂CH₂OH | —H | —CH₂OH |
| 27 | —H | —H | —CH₂OH |
| 28 | —H | —H | —CH₂OCH₂CH₃ |
| 29 | —H | —H | —CH₂OCH(CH₃)₂ |
| 30 | —CH₂CH₂CH₂OH | —H | —CH(OH)CH₃ |
| 31 | —CH₂CH₂CH₂OH | —H | —CH(OH)CH₂CH₃ |
| 32 | —H | —H | —CH(OH)CH₃ |
| 33 | —H | —H | (+/−)—CH(OCH₃)CH₃ |
| 34 | —CH₂CH₂CH₂OH | —CH₂OH | CH₂OCH(CH₃)₂ |

13. The method of claim 7 wherein said indenocarbazole derivative has the formula:

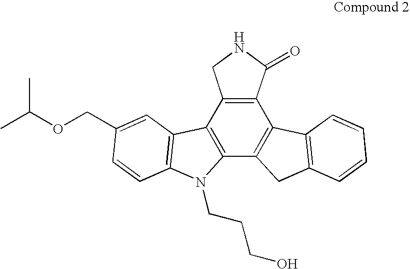

14. The method of claim 7 wherein said indenocarbazole derivative has the formula:

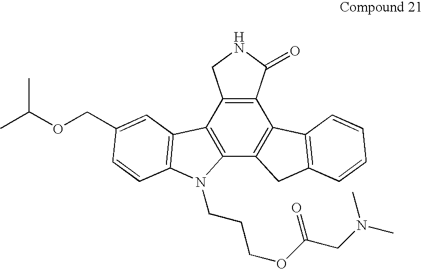

15. The method of claim 7 wherein the fused indenocarbazole forms part of a pharmaceutical composition comprising a pharmaceutical solution or suspension, a delivery device, or a drug-eluting polymer matrix that is sized and configured to be injected or inserted into the eye of a patient.

16. A method of treating glaucoma, comprising administering to a patient a therapeutically effective amount of an indenocarbazole derivative having the formula III, or a salt or prodrug thereof: 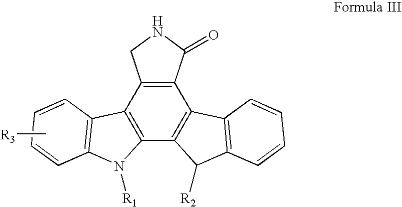
wherein:
R1 and R2 are the same or different and are independently selected from H, or alkyl of 1-8 carbons, substituted with OH, or —OR4 where R4 is an alkyl of 1-4 carbons, aryl of 6-12 carbons, or the residue of an amino acid after the hydroxyl group of the carboxyl group is removed; and
R3 is —CH₂OH; —CH₂OR7; —(CH₂)ₙSR5; —(CH₂)ₙSO_{y}R5; —CH₂SR₅; or alkyl of 1-8 carbons, substituted with —OH, —OR5, —OR8, —CH₂OR7, —SO_{y}R6 or —SR6; and wherein
R5 is alkyl of 1-4 carbons or aryl of 6-12 carbons;
R6 is H, alkyl of 1-4 carbons, aryl of 6-10 carbons or heteroaryl of 5-12 ring members;
R7 is H or alkyl of 1-4 carbons;
R8 is the residue of an amino acid after the hydroxyl group of the carboxyl group is removed;
n is an integer of 1-4; and
y is 1 or 2.
